(19) United States
(12) Patent Application Publication
(10) Pub. No.: US 2004/0204730 A1
(43) Pub. Date: Oct. 14, 2004

(54) Surgical valve cutter

(76) Inventors: Mark C. Goldberg, Sharon, MA (US); Lev Melinyshyn, Buffalo Grove, MA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/408,963

(22) Filed: Apr. 8, 2003

(51) Int. Cl. A61B 17/32

(52) U.S. Cl. 606/170; 606/167; 606/110

(57) ABSTRACT

A surgical valvulotome has a detachable hub. The hub has a hub body and a removable cap. A seal disposed between the hub body and the cap holds an end of a catheter. Tapering side walls squeeze the seal when the hub body and cap are tightened. Loosening the cap allows the seal to return to its original shape, releasing the catheter.

### BACKGROUND OF THE INVENTION

The present invention relates to surgical instruments and more particularly to surgical valve cutters (valvulotomes) used to cut the leaflets on a venous valve so that the vein can be used to bypass an artery.

The saphenous vein is a major vessel used in in-situ arterial bypasses, in which blood flow is directed in a reverse direction through the vein as it lies anatomically within the body. The vein naturally has venous valve leaflets that function as one-way valves against such flow. Accordingly, the leaflets must be removed or disabled before the vein can be used in the in-situ procedure. Conventionally, the leaflets are removed by moving a valvulotome through the vein to incise the valve leaflets.

Valvulotomes and the in-situ bypass procedure are described in U.S. Pat. Nos. 5,601,580 and 5,304,189, the disclosure of which is incorporated herein by reference. In general, valvulotomes include a valve-cutting head attached to a catheter that passes through the vein. The cutting head includes blades on its trailing edge that incise the valve leaflets as the cutting head passes through the valve. The catheter enables the cutting head to be operated from a remote handle.

One difficulty with the in-situ procedure arises from the fact that the vein has a series of side branches. These side branches collect blood and deposit blood into the vein. During the in-situ procedure, a valvulotome can sometimes inadvertently pass into one of the side branches, where it can become trapped. Once a cutting head has entered a side branch, the blades make it difficult to pull the cutting head back into the main channel without the blades damaging the junction of the side branch and the vein. One option for addressing the problem of the cutting head entering a side branch is to withdraw the cutting head through the wall of the side branch. This, however, requires severing the catheter, effectively destroying the valvulotome.

### SUMMARY OF THE INVENTION

The present invention provides an alternative solution to the problem of removing a cutting head from a side branch. The cutting head on the disclosed device can be removed from the side branch without either pulling the cutting head back into the main channel or severing the catheter.

Like previously-known valvulotomes, the surgical valve cutter of the present invention comprises a valve-cutting head attached to a distal end of a catheter. It also has a hub that provides access to the lumen of the catheter at its proximal end. However, unlike prior known devices, the hub is detachably connected to the proximal end of the catheter. Thus, if the cutting head becomes trapped in a side branch, the hub can be removed, and the cutting head can be removed through the wall of the side branch without the need to sever the catheter or destroy the device.

In one embodiment of the invention, the proximal end of the catheter fits within a passageway in a compressible seal between the hub body and a removable cap. The diameter of the passageway is just slightly larger than the outside diameter of the catheter. The seal is disposed within tapering interior walls, and has an outside diameter that is greater than the narrowest inside diameter of the tapering interior walls. Tightening the cap to the hub compresses the seal within the tapering walls, causing the seal to hold the catheter tightly. In the event that the physician wishes to detach the catheter from the hub body, he or she merely needs to loosen the cap. Loosening of the cap allows the seal to return to its original shape, releasing the catheter and allowing the cutting head to be removed without the need to pull it through the junction of the vein and the side branch.

### BRIEF DESCRIPTION OF THE DRAWINGS

FIG. 1 is a plan view of one embodiment of a valvulotome in accordance with the present invention;

FIG. 2 is an enlarged view of the cutting head and leader of the valve cutter of FIG. 1;

FIG. 3 is a cross-sectional view of the hub and a portion of the catheter of the valvulotome of FIG. 1; and

FIG. 4 is an exploded cross-sectional view of the hub and catheter of FIG. 3.

### DETAILED DESCRIPTION OF THE DRAWINGS

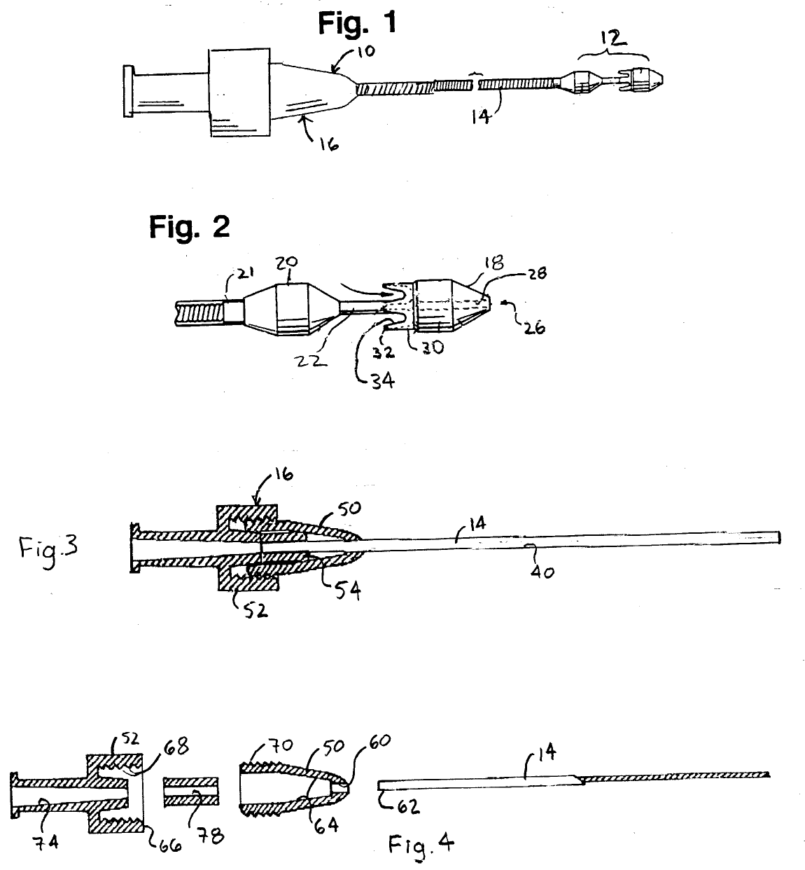

One embodiment of a surgical valve cutter or valvulotome 10 in accordance with the present invention can be seen in FIG. 1. Although the valvulotome is discussed below in connection with in-situ bypass procedures, it is not limited to such procedures and may be applied to any vascular operation requiring a non-reversed vein graft. Such applications may, for example, be found during distal infrainguinal bypasses when a non-anatomic position is required (ex: pro funda femoris to anterior tibial artery), composite vein infrainguinal bypasses, or even an aorta-renal bypass.

The valvulotome 10 that has been illustrated includes a cutting head 12, a catheter 14, and a hub 16. Each of these parts will be discussed in detail below. In general, the valvulotome is used by introducing the cutting head into a vein, adding fluid to dilate and lubricate the vein and open the valve leaflets, and then advancing at least part of the cutting head through the valve. Irrigation pressure or blood pressure is then used to close the valve, and the direction of the cutting head is reversed. As the cutting head is retracted, edges on the cutting head incise the leaflets on the valve.

The cutting head 12 that has been illustrated in FIG. 2 is a conventional one that includes a cutter 18, a leader 20, and a stem 22 between the cutter and the leader. The cutter may be made of any material that is safe for use in the body and is capable of taking and holding a knife edge. Stainless steel is preferred for the fabrication of the cutter. The valvulotome 10 may, for example, include 1.5 mm, 2.0 mm, 2.5 mm, 3.0 mm, 3.5 mm, and 4.0 mm or other size diameter cutters. The choice of cutter size is a matter of judgment, although it is recommended that a size smaller than the vein be employed.

The leading end of the illustrated cutter 18 is in the shape of a cone that has been truncated and bored at its distal tip to provide an irrigation port 26 that communicates with a central lumen 28. The opposite end of the cutter has prongs 30 with sharp cutting blades 32 with side edges 34. In use, the blades of the prongs initially pierce the valve leaflets as the cutter begins to re-enter the valve, and the side edges shear of the leaflets as the cutter is pulled through to gently widen the initial incisions until the entire valve is cleanly cored out and captured in the cutter. Other arrangements of blades could be used.

The use of the leader 20 is preferred but not required in the practice of the invention. The illustrated leader is used to connect the cutter 18 to a distal end 21 of the catheter 14. A fiber-optic bundle may also be mounted in the leader to enable the surgeon to visually monitor the action of the prongs 30 as they cut each successive valve.

The illustrated leader 20 is attached to the cutter 18 using the rigid stem 22, which is centered on the axis of both the cutter and the leader. The stem provides an open lumen from the irrigation port 26 to the leader. A spring may also be used as the stem, providing additional irrigation through the spacings between the coils of the spring. The stem 22 should be of a length sufficient to permit the valve leaflets to close (i.e., clear the leader when the cutting head is being retracted) before meeting the blades 32 on the cutter 18.

The surgeon controls the valvulotome 10 from the hub 16, seen in FIGS. 3 and 4. The hub is remotely connected to the cutting head 12 by the catheter 14. Using the catheter, the surgeon can cause the cutting head 12 to advance, withdraw, or rotate within the vein by pushing, pulling, or rotating the hub.

The catheter 14 includes a lumen 40 through which fluids can be supplied for irrigating the vein adjacent to the cutting head 12. The catheter can also be used to carry a fiber-optic element. The illustrated catheter is in the form of a hollow tube. The catheter can alternatively take the form of a tightly wound coil spring that is covered with an inextensible sheath. Such a coil spring would preferably be made of stainless steel, while the sheath would preferably be made of a low-surface-friction thromboresistant material such as polyurethane.

The illustrated hub 16 includes three parts: a hub body 50, a locking cap 52, and a seal 54. The hub provides a port that enables the surgeon to irrigate the vein while the valvulotome is being used. Unlike in previously known devices, the hub has a detachable construction, described below. Thus, in the event the cutting head inadvertently enters a side branch of the vein, the hub can be detached, allowing the cutting head and catheter to be withdrawn through an incision in the side branch without the need to sever the catheter.

The hub body 50 that is shown in FIGS. 3 and 4 is generally conical, although it could have other shapes. The hub body has an end aperture 60 through which a proximal end 62 of the catheter 14 passes. The illustrated end aperture has a diameter of approximately 1.37 mm for use with a 1.35 mm catheter. The illustrated hub body has tapering interior walls 64. Near the end aperture, the walls are about 3.91 mm apart. Approximately 9.96 mm away from the aperture, the walls are about 5.11 apart, tapering at an angle of between 10 and 20 degrees. Other sizes and arrangements can be used for other circumstances.

The locking cap 52 is removably secured against the hub body 50. In the illustrated embodiment of the invention, the cap has a peripheral wall 66 with internal thread segments 68 that engage external threads 70 on the hub body. Other arrangements can be used to connect the cap and the hub body. The illustrated cap 52 includes a central fluid channel segment 74 with a conventional luer taper. The luer feature enables a surgeon to attach a syringe to the valvulotome 10, providing fluid communication with the lumen 40 of the catheter 14.

The illustrated compressible seal 54 is disposed between the cap 52 and the hub body 50, a portion of the seal being disposed within the tapering interior walls 64. The illustrated seal is made of silicone, although other materials could also be used. The seal has an outside diameter that is greater than the narrowest inside diameter of the tapering interior walls. As illustrated, the seal is about 9.96 mm long and has a diameter of approximately 4.95 mm. The seal also includes a central passageway 78 that has a diameter that is just slightly larger than the outside diameter of the proximal end 62 of the catheter 14. The illustrated passageway has a diameter of approximately 1.37 mm. Other arrangements could be used.

In assembling the valvulotome 10, the catheter 14 is threaded through end aperture 60 in the hub body 50 and through passageway 78 in the seal 54. The seal is then radially squeezed when the cap 52 is tightened to the hub body. Squeezing the seal reduces the diameter of the passageway, securing the catheter in place within the hub 16.

In the event it is necessary to detach the hub 16 from the catheter 14, the cap 52 and the hub body 50 can be unscrewed, permitting the seal 54 to return to an uncompressed state and releasing the catheter.

This description of one embodiment of the invention has been provided merely for illustrative purposes. The scope of the invention is set forth in the following claims.

1) A surgical valve cutter comprising:
a cutting head;
a catheter with a lumen, a proximal end, and a distal end, the distal end being attached to the cutting head; and
a hub that provides access to the lumen of the catheter and is detachably connected to the proximal end of the catheter.

2) A surgical valve cutter comprising:
a cutting head;
a catheter with a lumen, a proximal end, and a distal end, the distal end being attached to the cutting head;
a hub body with an end aperture through which the proximal end of the catheter passes;
a cap that is removably secured against the hub body;
a compressible seal between the hub body and the cap, the seal being compressed when the cap is secured against the hub body; and
a passageway in the seal that holds the proximal end of the catheter tightly when the seal is in a compressed state, and which releases the proximal end of the catheter when the seal is in an uncompressed state.

3) A surgical valve cutter comprising:
a cutting head;
a catheter with a lumen, a proximal end, and a distal end, the distal end being attached to the cutting head;
a hub body with an end aperture through which the proximal end of the catheter passes;
a cap that is removably secured against the hub body;
a compressible seal that is disposed within tapering interior walls of the hub body, the seal having an outside diameter that is greater than the narrowest inside diameter of the tapering interior walls; and
a passageway in the seal that has a diameter that is just slightly larger than the outside diameter of the proximal end of the catheter.
